(19) United States
(12) Patent Application Publication
(10) Pub. No.: US 2022/0267865 A1
(43) Pub. Date: Aug. 25, 2022

(54) IN VITRO METHOD FOR THE DIAGNOSIS OF VIRAL INFECTIONS

(71) Applicants: UNIVERSIDADE DE SANTIAGO DE COMPOSTELA, Santiago de Compostela (ES); SERVIZO GALEGO DE SAÚDE, Santiago de Compostela (ES)

(72) Inventors: Ruth BARRAL ARCA, SANTIAGO DE COMPOSTELA (ES); Federico MARTINÓN TORRES, SANTIAGO DE COMPOSTELA (ES); Antonio SALAS ELLACURIAGA, SANTIAGO DE COMPOSTELA (ES)

(21) Appl. No.: 17/427,844

(22) PCT Filed: Feb. 5, 2020
(86) PCT No.: PCT/EP2020/052907

§ 371 Date: Aug. 2, 2021

(30) Foreign Application Priority Data
Feb. 5, 2019 (EP) 19382084.2

(51) Int. Cl. C12Q 1/70

(52) U.S. Cl. CPC C12Q 1/70

(57) ABSTRACT

In vitro method for the diagnosis of viral infections. The present invention refers to an in vitro method for the diagnosis of viral infections, for selecting a therapy for a patient suffering a viral infection and/or for monitoring the response of vaccinated patients to a viral vaccine.

### FIELD OF THE INVENTION

The present invention refers to the medical field. Particularly, it refers to an in vitro method for the diagnosis of viral infections, for selecting a therapy for a patient suffering a viral infection and/or for monitoring the response of vaccinated patients to a viral vaccine.

### STATE OF THE ART

There have been increasing efforts to find host biomarkers to identify viral infections in febrile children. The interest stems from the need to avoid the antibiotics overuse which is accelerating antimicrobial resistance worldwide and has been declared as one of the greatest threats to human health by the World Health Organization (WHO).

In the last years the employment of host blood gene expression biomarkers, derived from transcriptomic studies, for identifying phenotypically similar diseases have experience an explosion as it has yielded promising results in scenarios where the available technology is uncertain or inefficient.

Up to the date, several signatures for different infectious diseases have been described but its implementation is still limited. For diagnostic tests based on RNA signatures to be translated into clinical set up, the first step is to identify a small number of transcripts able to identify the disease in question with enough precision. The second requisite is to develop a fast and cheap method or protocol for measure the gene expression levels such as qPCR or new emerging technologies which may hold the key to the introduction of transcriptomic biomarkers into mainstream clinical decision-making in the next years.

It is herein provided significant results about the performance of a 2-transcript host RNA signature for discriminating viral infections which hold the potential to be used in mainstream clinical decision making.

### DESCRIPTION OF THE INVENTION

### Brief Description of the Invention

The present invention is focused on solving the above cited problems and, after the study and analysis of transcriptome modifications, it is herein provided an in vitro method for the diagnosis of viral infections, for selecting a therapy for a patient suffering a viral infection and/or for monitoring the response of vaccinated patients to a viral vaccine.

The two-transcript signature proposed in the present invention is able to distinguish viral infections in a broad sense. Therefore, it can be used for the diagnosis of viral infections, for selecting a therapy for a patient suffering a viral infection and/or for monitoring the response of vaccinated patients to a life attenuated viral vaccine.

The diagnose signature is based on assigning to each patient a disease risk score calculated adding the total intensity of both transcripts following the formula:

Disease Risk Score=log(expression [ENSG00000273149])+log(expression [ENSG00000254680])

Lower scores imply viral assignment, whereas higher scores correspond to healthy assignment. The optimal threshold value is defined by the Youden's J statistic, as the point of the ROC curve that maximizes the specificity and the sensitivity.

Although in a preferred embodiment the present invention refers to a RNA signature which comprises, in combination, the SEQ ID NO: 1 (ENSG00000273149) and SEQ ID NO: 2 (ENSG00000254680), it is important to note that the present invention can be carried out by using one of the above cited RNAs. Thus, in a preferred embodiment, the present invention can be carried out by using SEQ ID NO: 1 (ENSG00000273149). Please refer to FIG. 1 wherein it is shown the AUC associated with the use of SEQ ID NO: 1 (ENSG00000273149) as biomarker in the context of the present invention. Alternatively, in a preferred embodiment, the present invention can be carried out by using SEQ ID NO: 2 (ENSG00000254680). Please refer to FIG. 2 wherein it is shown the AUC associated with the use of SEQ ID NO: 2 (ENSG00000254680) as biomarker in the context of the present invention. Finally, as explained above, in a preferred embodiment, the present invention can be carried out by using SEQ ID NO: 1 (ENSG00000273149) in combination with SEQ ID NO: 2 (ENSG00000254680). Please refer to FIG. 3 wherein it is shown the AUC associated with the use of SEQ ID NO: 1 (ENSG00000273149) in combination with SEQ ID NO: 2 (ENSG00000254680) as biomarker signature in the context of the present invention.

The RNA transcriptomic signature of the invention is suitable for distinguishing vaccinated from unvaccinated children and children affected by community acquired Rotavirus. Consequently, this signature could be used to detect vaccinated failures and prevent severe Rotavirus re-infections. However, surprisingly, the biomarkers and signature provided by the present invention are able to distinguish healthy controls from viral infections in a broad sense including (non-exhaustive list): Bocavirus, Influenza, Metaneumovirus, Respiratory Syncytial virus and Varicella Zoster virus (see FIGS. 1I, 1J, 2C and 2G).

According to the ROC curves shown in FIGS. 1, 2 and 3, the RNA of SEQ ID NO: 1, the RNA of SEQ ID NO: 2, or the combination of both RNAs, has an extremely high sensitivity, able to classify as viral infections children and cells exposed to live attenuated vaccines such as Rotateq® and Varivax®. The high sensitivity of these biomarkers will be of particularly interest in kindergartens and hospitals where Rotavirus can easy become endemic causing serious health problems.

The fact that the RNAs of SEQ ID NO: 1 and/or SEQ ID NO: 2 have been found differentially expressed between vaccinated-or-wildtype infected children and healthy controls, showing a high sensitivity, can be considered as an unexpected and promising result. So, the above cited RNAs can be efficiently used for the diagnosis of viral infections, for selecting a therapy for a patient suffering a viral infection and/or for monitoring the response of vaccinated patients to a viral vaccine.

Particularly, Table 1 shows the AUC, sensitivity and specificity associated with the use of SEQ ID NO: 1 (ENSG00000273149) for the identification of viral or bacterial infections. Such as it can be observed in Table 1 the use of SEQ ID NO: 1 for the identification of variety of viral infections gives rise to an AUC higher than 0.9, with a sensitivity and specificity higher than 0.8. In contrast, the use of SEQ ID NO: 1 for the identification of bacterial infections gives rise to an AUC lower than 0.8, with a sensitivity and/or specificity lower than 0.8.

TABLE 1

| | Data base | | | | | |
|---|---|---|---|---|---|---|
| Pathogen | reference | Comparisson | n | AUC | Sensitivity | Specificity |
| Influenzavirus A | PRJNA230906 | virus vs healthy | 4 | 1 | 1 | 1 |
| (H7N9) China | | control | | | | |
| Rotavirus | GSE69529 | virus vs healthy | 115 | 0.932 | 0.85 | 0.82 |
| | | control | | | | |
| Varicella zoster | GSE121385 | virus vs healthy | 6 | 1 | 1 | 1 |
| | | control | | | | |
| Rotavirus Spain | PRJNA325575 | virus vs healthy | 18 | 1 | 1 | 1 |
| | | control | | | | |
| Bocavirus | Own data | virus vs healthy | 7 | 1 | 1 | 1 |
| | unpublished | control | | | | |
| Metapneumovirus | Own data | virus vs healthy | 7 | 1 | 1 | 1 |
| | unpublished | control | | | | |
| Rhinovirus | Own data | virus vs healthy | 8 | 1 | 1 | 1 |
| | unpublished | control | | | | |
| Respiratory syncytial | Own data | virus vs healthy | 41 | 1 | 1 | 1 |
| virus (RSV) | unpublished | control | | | | |
| Bacteria | GSE69529 | bacteria vs healthy | 175 | 0.761 | 0.70 | 0.77 |
| | | control | | | | |
| Rotavirus | GSE69529 | bacteria vs rotavirus | 220 | 0.797 | 0.85 | 0.63 |

Statistical results associated with the use of SEQ ID NO: 1 (ENSG00000273149)

On the other hand, Table 2 shows the AUC, sensitivity and specificity associated with the use of the combination of SEQ ID NO: 1 (ENSG00000273149) and SEQ ID NO: 2 (ENSG00000254680) for the identification of viral or bacterial infections. Such as it can be observed in Table 2 the use of SEQ ID NO: 1 and SEQ ID NO: 2 for the identification of a variety of viral infections gives rise to an AUC higher than 0.89, with a sensitivity and specificity higher than 0.8. In contrast, the use of SEQ ID NO: 1 and SEQ ID NO: 2 for the identification of bacterial infections gives rise to an AUC lower than 0.8, with a sensitivity and/or specificity lower than 0.8.

TABLE 2

| | Data base | | | | | |
|---|---|---|---|---|---|---|
| Pathogen | reference | Comparisson | n | AUC | Sensitivity | Specificity |
| Influenzavirus A | PRJNA230906 | virus vs healthy | 4 | 1 | 1 | 1 |
| (H7N9) China | | control | | | | |
| Dengue | GSE98859 | virus vs healthy | 5 | 1 | 1 | 1 |
| | | control | | | | |
| Enterovirus | GSE94551 | virus vs healthy | 6 | 1 | 1 | 1 |
| | | control | | | | |
| Rotavirus Spain | PRJNA325575 | virus vs healthy | 18 | 1 | 1 | 1 |
| | | control | | | | |
| Enterovirus + Rotavirus | GSE69529 | virus vs healthy | 115 | 0.891 | 0.857 | 0.837 |
| Mexico | | control | | | | |
| Enterovirus + Rotavirus | GSE69529 | virus vs bacteria | 220 | 0.731 | 0.814 | 0.600 |
| Mexico | | | | | | |
| Enterovirus + Rotavirus | GSE69529 | bacteria vs healthy | 175 | 0.707 | 0.650 | 0.742 |
| Mexico | | control | | | | |
| Varicella zoster | GSE121385 | virus vs healthy | 6 | 1 | 1 | 1 |
| | | control | | | | |

Statistical results associated with the use of the combination of SEQ ID NO: 1 (ENSG00000273149) and SEQ ID NO: 2 (ENSG00000254680)

Particularly, the first embodiment of the present invention refers to an in vitro method for the diagnosis of viral infections in a patient which comprises determining the level of at least the RNA of SEQ ID NO: 1 and/or SEQ ID NO: 2, or a protein encoded thereof, in a biological sample obtained from the patient, wherein a reduced level of at least the RNA of SEQ ID NO: 1 and/or SEQ ID NO: 2, or the protein encoded thereof, as compared with the reference level determined in healthy control subjects, preferably as compared with a corresponding predetermined threshold level selected to provide a sensitivity and specificity of at least 0.8, is an indication that the patient is suffering from a viral infection.

The second embodiment of the present invention refers to an in vitro method for selecting a therapy for a patient which comprises determining the level of at least the RNA of SEQ ID NO: 1 and/or SEQ ID NO: 2, or a protein encoded thereof, in a biological sample obtained from the patient, wherein a reduced level of at least the RNA of SEQ ID NO: 1 and/or SEQ ID NO: 2, or the protein encoded thereof, as compared with the reference level determined in healthy control subjects, preferably as compared with a corresponding predetermined threshold level selected to provide a sensitivity and specificity of at least 0.8, is an indication that the patient is suffering from a viral infection and consequently a treatment with antibiotics can be discarded.

The third embodiment of the present invention refers to an in vitro method for monitoring the response of vaccinated patients to a viral vaccine which comprises determining the level of at least the RNA of SEQ ID NO: 1 and/or SEQ ID NO: 2, or a protein encoded thereof, in a biological sample obtained from the patient, wherein a reduced level of at least the RNA of SEQ ID NO: 1 and/or SEQ ID NO: 2, or the protein encoded thereof, preferably as compared with a corresponding predetermined threshold level selected to provide a sensitivity and specificity of at least 0.8, as compared with the reference level determined in healthy control subjects, is an indication that the patient is responding to the viral vaccine.

The fourth embodiment of the present invention refers to the in vitro use of at least the RNA of SEQ ID NO: 1 and/or SEQ ID NO: 2, or a protein encoded thereof, for the diagnosis of a viral infection in a patient.

The fifth embodiment of the present invention refers to the in vitro use of at least the RNA of SEQ ID NO: 1 and/or SEQ ID NO: 2, or a protein encoded thereof, for selecting a therapy for a patient with a viral infection.

The sixth embodiment of the present invention refers to the in vitro use of at least the RNA of SEQ ID NO: 1 and/or SEQ ID NO: 2, or a protein encoded thereof, for monitoring the response of vaccinated patients to a viral vaccine.

The seventh embodiment of the present invention refers to the in vitro use of a kit comprising reagents for the determination of the level of at least the RNA of SEQ ID NO: 1 and/or SEQ ID NO: 2, for the diagnosis of a viral infection, for selecting a therapy for a patient with a viral infection or for monitoring the response of vaccinated patients to a viral vaccine.

The eight embodiment of the present invention refers to a method for treating a patient which comprises selecting a therapy by determining the level of at least the RNA of SEQ ID NO: 1 and/or SEQ ID NO: 2, or a protein encoded thereof, in a biological sample obtained from the patient, wherein a reduced level of at least the RNA of SEQ ID NO: 1 and/or SEQ ID NO: 2, or the protein encoded thereof, as compared with the reference level determined in healthy control subjects, is an indication that the patient is suffering from a viral infection and consequently a treatment with antibiotics can be discarded, and wherein a higher level of at least the RNA of SEQ ID NO: 1 and/or SEQ ID NO: 2, or the protein encoded thereof, as compared with the reference level determined in healthy control subjects, is an indication that the patient is not suffering from a viral infection and consequently a treatment with antibiotics might be recommended.

In a preferred embodiment, the viral infection detected and/or treated according to the present invention is caused by (non-exhaustive list): Rotavirus, Varicella, Bocavirus, Influenza, Metapneumovirus, Rhinovirus or Respiratory syncytial virus.

In a preferred embodiment, the viral vaccine that has been used to treat the patient is a vaccine for the prophylactic treatment of a viral infection caused by non-exhaustive list): Rotavirus, Varicella, Bocavirus, Influenza, Metapneumovirus, Rhinovirus or Respiratory syncytial virus.

In a preferred embodiment, the present invention comprises determining the level of at least the RNA of SEQ ID NO: 1 in combination with the RNA of SEQ ID NO: 2, or proteins encoded thereof.

In a preferred embodiment, the present invention is carried out in a sample selected from the list: blood, serum, plasma or dermal fibroblasts.

For the purpose of the present invention the following terms are defined:
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.
The term “reference control level”, when referring to the level of the RNA biomarkers described in the present invention, refers to the level observed in healthy control subjects, which are not suffering a viral infection. The patient is likely to be infected with virus with a given sensitivity and specificity if the levels of the RNA biomarkers in the patient are blow said “reference control level”. A “reference” value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Preferably, the person skilled in the art may compare the RNA levels obtained according to the method of the invention with a defined threshold value. Furthermore, retrospective measurement of the RNA levels (or scores) in properly banked historical subject samples may be used in establishing these threshold values. Typically, the optimal sensitivity and specificity (and so the threshold value) can be determined using a Receiver Operating Characteristic (ROC) curve based on experimental data. For example, after determining the RNA levels in a group of reference, one can use algorithmic analysis for the statistic treatment of the measured concentrations of biomarkers in biological samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: R package pROC 1.13.0,MedCalc 9.2.0.1 medical statistical software, SPSS 9.0.

### DESCRIPTION OF THE FIGURES

FIG. 1. Classification for RNA of SEQ ID NO: 1 (ENSG00000273149). Classification performance based on the transcript ENSG00000273149 considering different viral pathogens and studies. A) Box and whisker plots of DRS for the external validation cohort PRJNA230906 (Chinese external validation cohort). B) Box and whisker plots of DRS for the external validation cohort GSE69529 (Mexican external validation cohort). C) Box and whisker plots of DRS for the external validation cohort PRJNA497243 (dermal fibroblast external validation cohort). D) Box and whisker plots of DRS for the discovery cohort (Spanish training cohort). E) ROC curve of DRS for the external validation cohort PRJNA230906 (Chinese external validation cohort; viral infection vs controls). F) ROC curves of DRS for the external validation cohort GSE69529 (Mexican external validation cohort; viral infection vs controls). G) ROC curve of DRS for the external validation cohort PRJNA497243 (dermal fibroblast external validation cohort; viral infection vs control). H) Receiver operating characteristic (ROC) curves of the discovery cohort (Spanish cohort performance; viral infection vs controls). I) Box and whisker plots of DRS for the External Spanish validation cohort (Spanish validation cohort). J) ROC curve of DRS for the External Spanish validation cohort (Spanish population). The Horizontal lines in boxes indicate median of the groups; the lower and upper edges of boxes interquartile ranges and the whiskers<1 times the interquartile range. On the X axis we have the sample status and on the Y axis the Disease Risk Score was calculated as the log2 of the transcript.

FIG. 2. Classification for RNA of SEQ ID NO: 2 (ENSG00000254680). Classification performance based on the transcript ENSG00000254680 considering different viral pathogens and studies. A) Receiver operating characteristic (ROC) curves of the discovery cohort (Spanish cohort performance; viral infection vs controls). B) Box and whisker plots of DRS for the external validation cohort PRJNA497243 (dermal fibroblast external validation cohort). C) Box and whisker plots of DRS for the External Spanish validation cohort (Spanish validation cohort). D) Box and whisker plots of DRS for the external validation cohort PRJNA230906 (Chinese external validation cohort). E) Box and whisker plots of DRS for the discovery cohort (Spanish training cohort). F) ROC curve of DRS for the external validation cohort PRJNA497243 (dermal fibroblast external validation cohort; viral infection vs control). G) ROC curve of DRS for the External Spanish validation cohort. H) ROC curve of DRS for the external validation cohort PRJNA230906 (Chinese external validation cohort; viral infection vs controls). I) Box and whisker plots of DRS for the external validation cohort GSE69529 (Mexican external validation cohort) validation cohort; viral infection vs controls. J) ROC curves of DRS for the external validation cohort GSE69529 (Mexican external validation cohort; viral infection vs controls). The Horizontal lines in boxes indicate median of the groups; the lower and upper edges of boxes interquartile ranges and the whiskers<1 times the interquartile range. On the X axis we have the sample status and on the Y axis the Disease Risk Score was calculated as the log2 of the transcript.

FIG. 3. Classification performance based on the 2-transcript disease risk score DRS combined as DRS=[log2(SEQ ID NO: 1)+log2(SEQ ID NO: 2)] considering different viral pathogens and studies. A) Box and whisker plots of DRS for the discovery cohort (Spanish training cohort). The Horizontal lines in boxes indicate median of the groups; the lower and upper edges of boxes interquartile ranges and the whiskers <1 times the interquartile range. On the X axis we have the sample status and on the Y axis the Disease Risk Score calculated as log2 of the sum of counts of our 2-transcript diagnosis model. B) Receiver operating characteristic (ROC) curves of the discovery cohort (Spanish cohort performance; viral infection vs controls). C) Box and whisker plots of DRS for the external validation cohort PRJNA230906 (Chinese external validation cohort). D) ROC curve of DRS for the external validation cohort PRJNA230906 (Chinese external validation cohort; viral infection vs controls). E) Box and whisker plots of DRS for the External Spanish validation cohort (Spanish validation cohort). F) ROC curve of DRS for the External Spanish validation cohort (Spanish validation cohort; viral infection vs controls. G) Box and whisker plots of DRS for the external validation cohort GSE69529 (Mexican external validation cohort). H) ROC curves of DRS for the external validation cohort GSE69529 (Mexican external validation cohort; viral infection vs controls). I) Box and whisker plots of DRS for the external validation cohort PRJNA497243 (dermal fibroblast external validation cohort). J) ROC curve of DRS for the external validation cohort PRJNA497243 (dermal fibroblast external validation cohort; viral infection vs control).

FIG. 4. Process used in the present invention for the validation of the SEQ ID NO: 1 and/or SEQ ID NO: 2 as reliable biomarkers for the diagnosis of viral infections.

### DETAILED DESCRIPTION OF THE INVENTION

### Example 1. Materials & Methods

### Example 1.1. Samples and Ethical Approval

All researchers were trained in the study protocol for patient recruitment, sample processing and sample storage. The study was conducted following the Good Clinical Practice. Written informed consent was obtained from a parent or legal guardian for each subject before study inclusion. The project was approved by the Ethical Committee of Clinical Investigation of Galicia (CEIC ref. 2012/301). Furthermore, this project followed the guidelines of the Declaration of Helsinki.

### Example 1.2. Spanish Cohort

46 samples: 6 controls (roughly 7 months of age with all the vaccines of the Spanish calendar up to date), 14 vaccinated (roughly 7 months of age with all the vaccines of the Spanish calendar up to date plus 3 Rotateq® dosis), 12 infected (with moderate and severe symptomatology) and 14 pre-vaccinated (children that had only received hepatitis B vaccine). 26 Western-European donors were prospectively collected at the Hospital Clinico Universitario of Santiago de Compostela (Galicia; Spain) during the period 2013 to 2014. Blood samples were obtained from these children using a PAXgene RNA tube (PreAnalytiX GmbH). All children recruited (ages ranging from nearly 2 to 34 months, male/female ratio=0.77) had routine immunization up-to-date. In wild type affected children the mean time elapsed from hospital admission to blood collection was three days, and in Rotavirus vaccinated children the blood sample was taken approximately a month after the last Rotateq® dose. There were no remarkable clinical features in the individuals recruited.

### Example 1.3. Mexican Cohort 77 samples of healthy and Rotavirus infected children were obtained from the NIH GEO repository accession number GSE69529.

### Example 1.4. Chinese Cohort

4 blood samples collected from patients with H7N9 infection (n=2) and healthy people (n=2). Sample were obtained from the NIH repository accession number PRJNA230906.

### Example 1.5. Varicella-Zoster fibroblast Cohort

6 samples of Varicella Zoster Virus (VZV)-infected human dermal fibroblasts cell line (HDF) infected with different strains or vaccines (Suduvax® and Varivix®) (n=1 control, n=2 wildtype strains and n=3 vaccinated) were obtained from NIH repository accession number PRJNA497243.

### Example 1.6. External Spanish Cohort

Validation cohort of children affected by viral infections of different etiologies was prospectively collected at the Hospital Clinico Universitario of Santiago de Compostela (Galicia; Spain) during the period 2013 to 2014. It comprises 1 Bocavirus patient, 2 Influenza patients, 1 Metapneumovirus, 2 Rhinovirus, 4 Rotavirus and 36 respiratory syncytial virus patients.

### Example 1.7. Bioinformatic Analysis

32 samples (6 controls, 14 rotaviruses vaccinated and 12 rotavirus infected children with moderate or severe symptomatology) of Western-European donors were prospectively collected at the Hospital Clinico Universitario of Santiago de Compostela (Galicia; Spain) during the period 2013 to 2014. A blood sample was obtained from these children using a PAXgene RNA tube (PreAnalytiX GmbH). There were no remarkable clinical features in the individuals recruited.

The quality standards followed in the present study were previously described in [Salas, A. et al., 2016. Strong down-regulation of glycophorin genes: A host defense mechanism against rotavirus infection. Infection, Genetics and Evolution, 44, 403-411]. Briefly, Bioanlayzer 2100 and Qubit 2.0 were employed to evaluate the quality and the quantity of the collected RNA. We used GLOBINclear™-Human Blood Globin Reduction Kit (Life Technologies; CA, USA) to eliminate globin mRNA and obtain a clearer signal from mRNAs from leukocytes. Poly(A)+mRNA fraction was isolated from total RNA, and cDNA libraries were obtained following Illumina's recommendations. An equimolar pooling of the libraries was performed before clusters generation using cbot from Illumina. An Illumina HiSeq 2000 sequencer was used to sequence the pool of cDNA libraries using paired-end sequencing (100×2).

First of all, we performed a quality control of the raw data using FastaQC (http://www.bioinformatics.babraham.ac.uk/projects/fastqc) and MultiQC to ensure that there were no problems or biases in our data which may affect the downstream analysis. Afterwards, the whole transcriptome paired-end reads were mapped against the version of the human genome provided by Ensembl (version GRCh37) using the ultrafast universal RNA-seq aligner STAR. We also used STAR for counting the number of reads that map to each gene.

The next step was the normalization of the count data to reduce the systematic technical effects that may appear in the data, and therefore decrease the technical bias impact on the final results. Currently many methods for normalizing RNA-seq data have been developed, however a gold standard normalization method has not been stablished yet. For normalizing the data, we used the statistical software R V3.4.3 (http:/www.r-project.org) and we tried several methods such as: RPKM Reads per million mapped reads, TMM implemented in edgeR package, CQN Conditional quantile normalization from tweeDEseq package and finally Deseq2 implemented in the package of the same name. All of the methods yielded virtually the same result, so we chose the normalization method included in the Deseq2 package, as this package was chosen for performing the downstream analysis.

Finally, we used the Negative Binomial distribution, implemented in the DESeq package together with the Surrogate Variable Analysis (SVA) method implemented in the sva R package for the estimation of the differentially expressed genes (DEG), between vaccinated children and healthy controls, and minimize batch effects between sequencing runs. A generalized linear model was fitted in each cohort, and at statistic was calculated for each gene, and then P-values obtained were corrected for multiple testing using the Benjamini-Hochberg false discovery rate approach. We obtained 8997 differentially expressed genes in comparison.

We applied a known variable selection algorithm called elastic net, to the genes differentially expressed (P-adjusted<0.05) between vaccinated and controls that have a log2change higher than two units using the glmnet R package. The parameters needed for the calculation of elastic net were estimated using 10-fold cross-validation. Obtaining an 18-transcript signature.

In order to determine a less complex signature we looked for the most informative genes between the ones previously selected by the Elastic net algorithm, using a machine learning approach a single-hidden-layer neural network model that was fitted with the R package nnet, obtaining a (SEQ ID NO: 1 and SEQ ID NO: 2) transcript signature:

Disease Risk Score=log (expression [ENSG00000273149])+log(expression [ENSG00000254680])

The performance of the proposed signature as potential diagnosis tools was evaluated using Receiver Operating Characteristic (ROC) curves that represent the true positive rate (TPR) against the false positive rate (FPR) at different threshold cut-points. ROC curves were built in R using the package pROC.

After finding this 2-transcript signature, we evaluated its performance with ROC curves created with the R package pROC.

Finally, we performed an external validation with different external datasets to evaluate if the discovered signal was specific for the rotavirus life attenuated virus that contains the vaccine or if it would be a viral signal in a broad sense and to assess the accuracy in truly independent datasets.

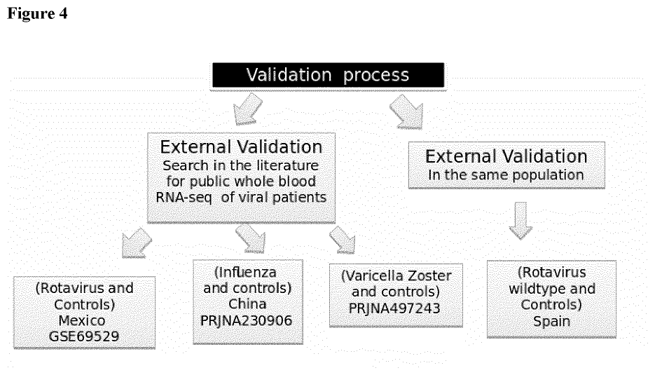

Following the strategy represented in FIG. 4 it was confirmed that this signature is able to distinguish healthy children from children undergoing a viral infection regardless of the virus. The performance was good even in virus from different families and that cause different phenotypes such as Rotavirus and Influenza.

### Example 2. Results

### Example 2.1. RNA-Seq Results

In order to study the changes experienced in the transcriptome of vaccinated children and children with community acquired Rotavirus, a large-scale expression screening was performed using a RNA-Seq approach. A comparison of gene expression between children with community acquired rotavirus and healthy controls indicates a total of 9544 genes show statistically significant differences, whereas 8997 genes showed statistically significant differences when comparing children vaccinated against rotavirus with controls.

### Example 2.2. RNA of SEQ ID NO: 1 or SEQ ID NO: 2 as Biomarkers for Diagnosis of Viral Infections

It was examined whether patients clustered according to their disease status (viral infection, bacterial infection and healthy controls) when employing only one of the two genes of the DRS.

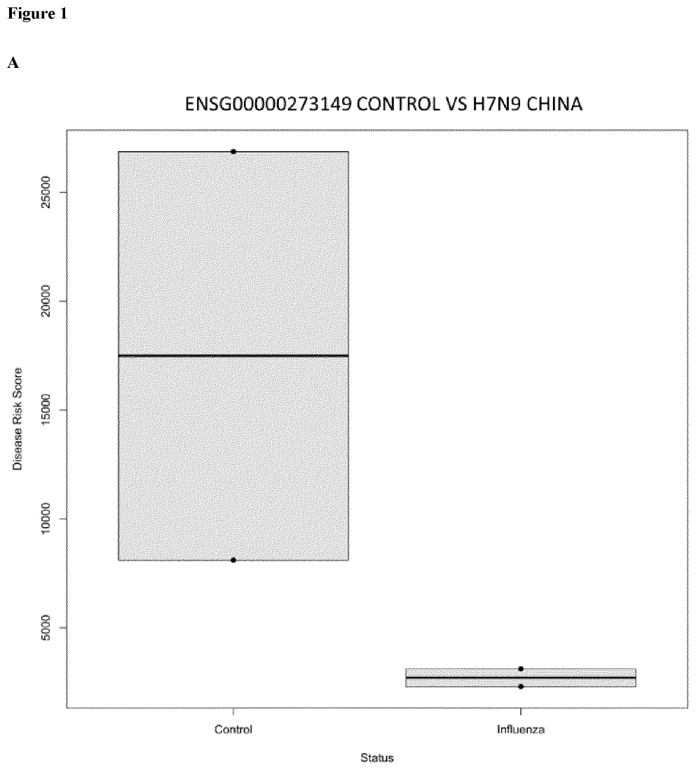

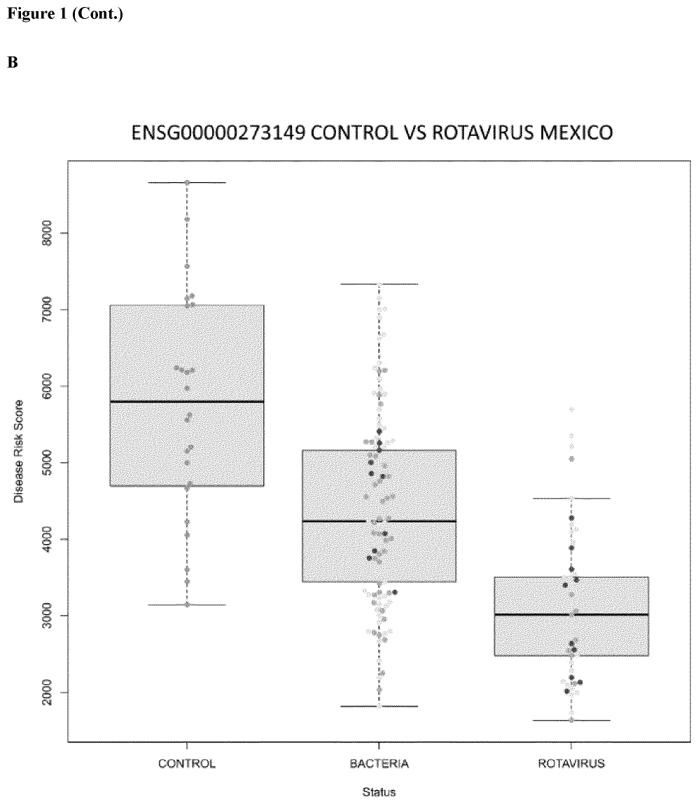

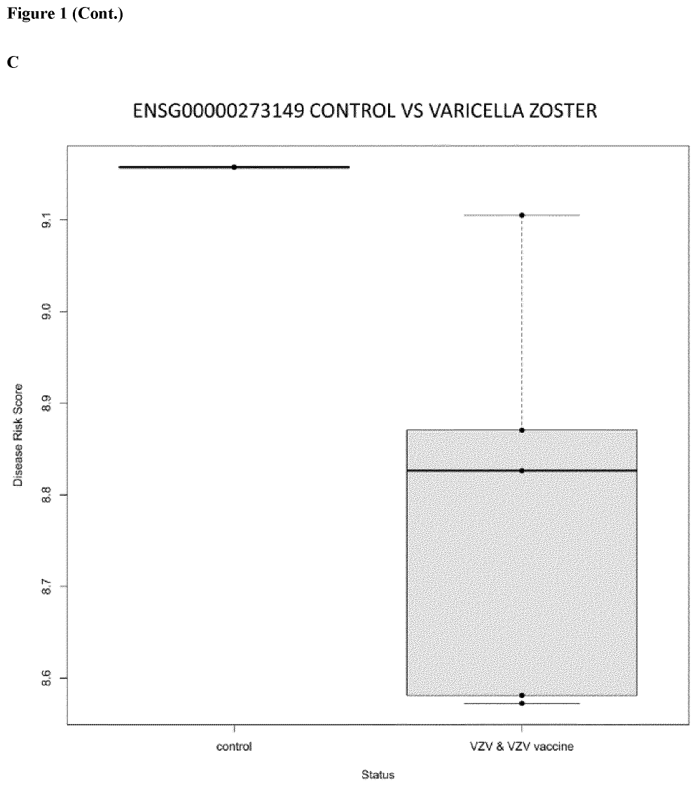

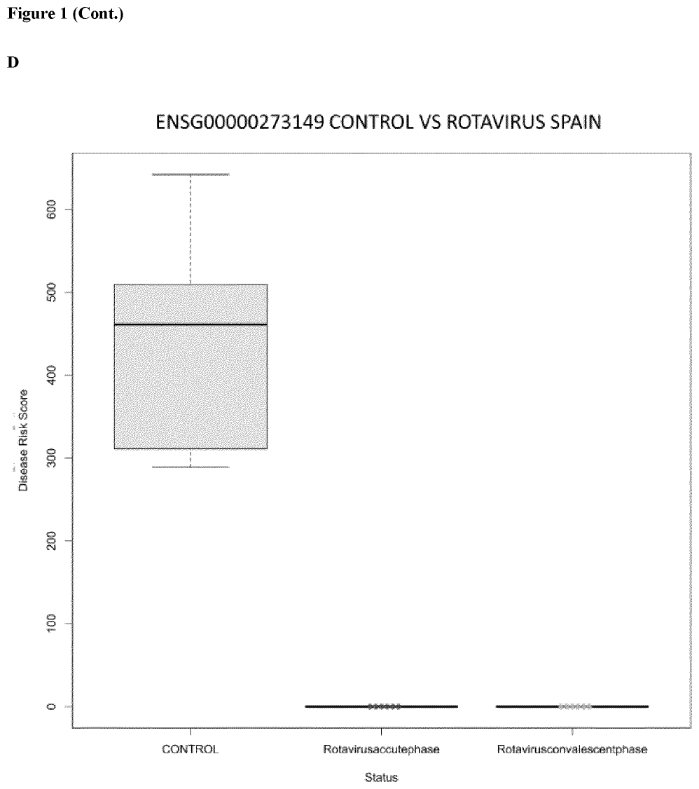

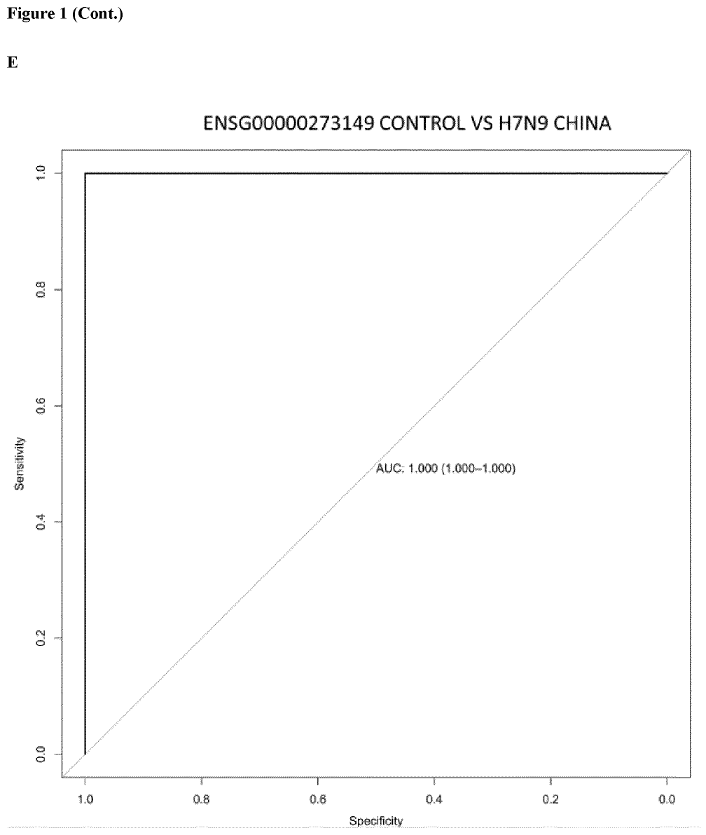

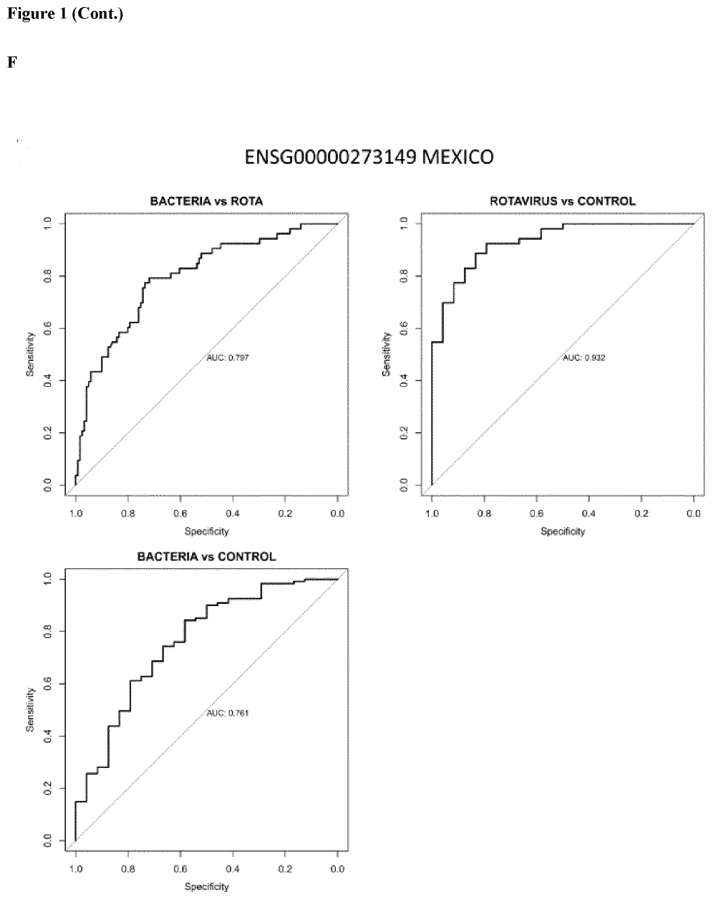

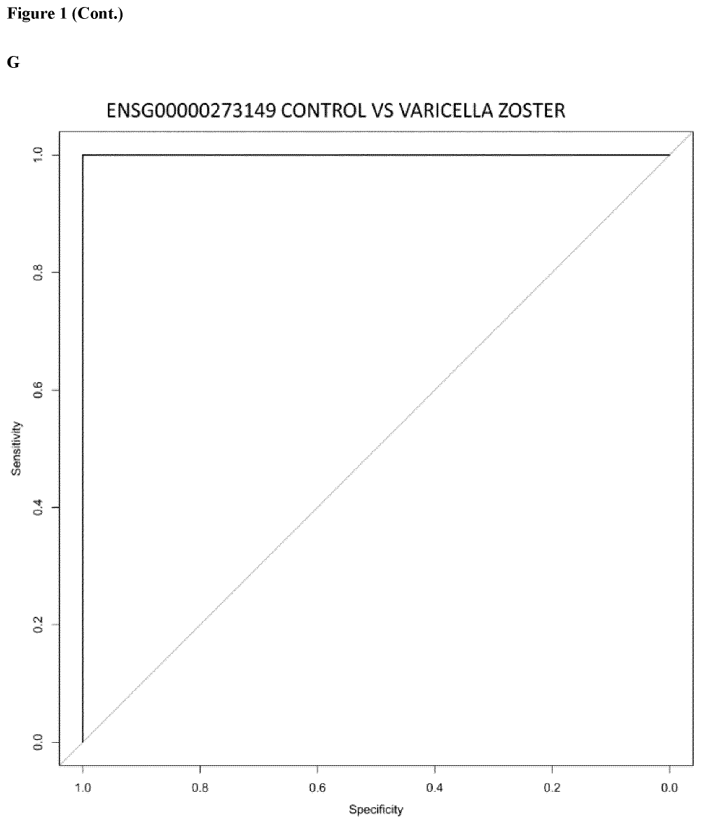

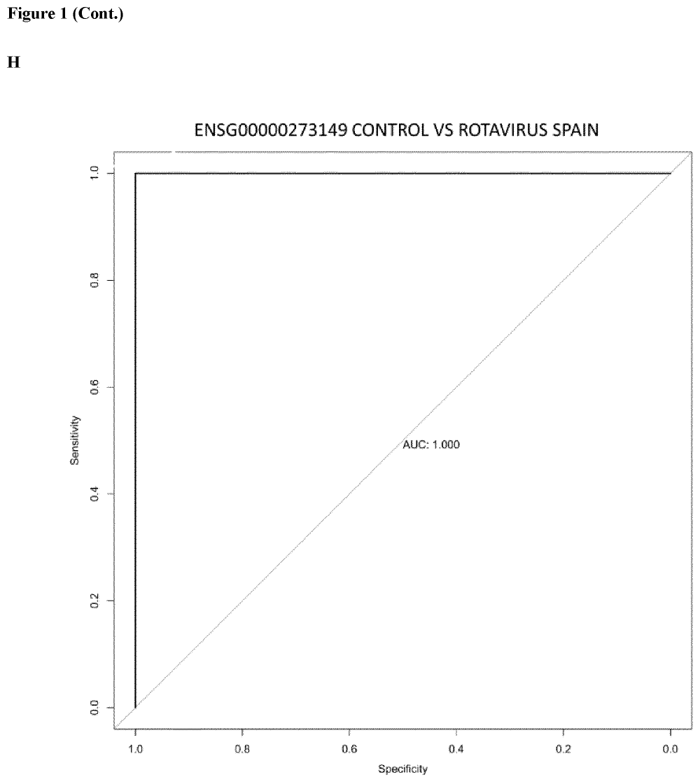

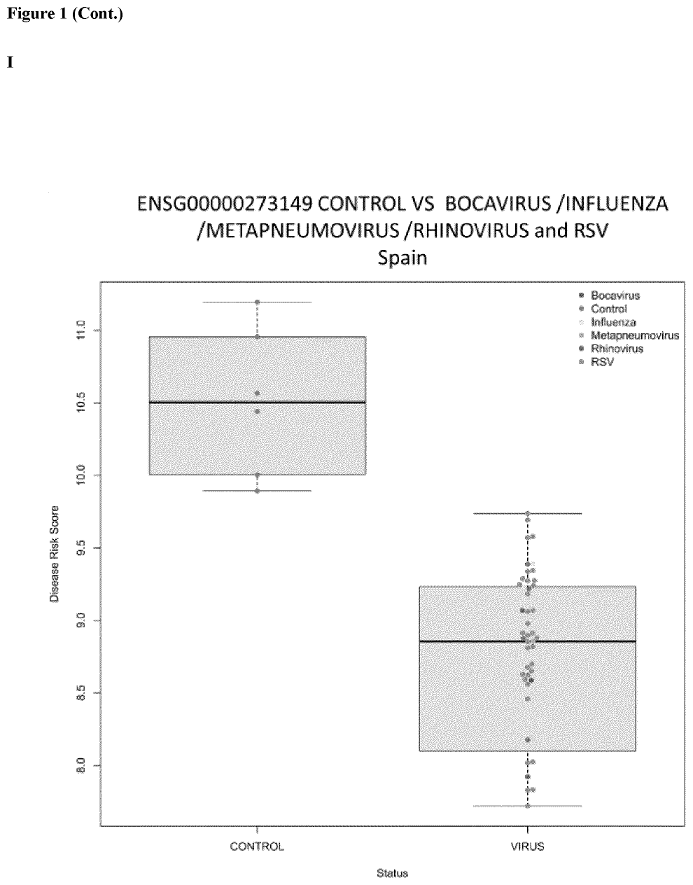

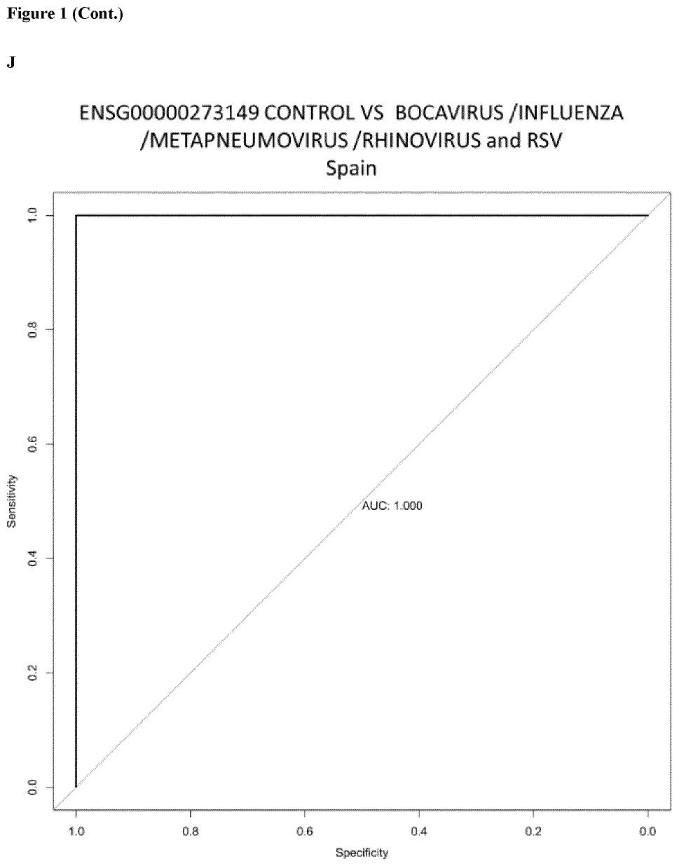

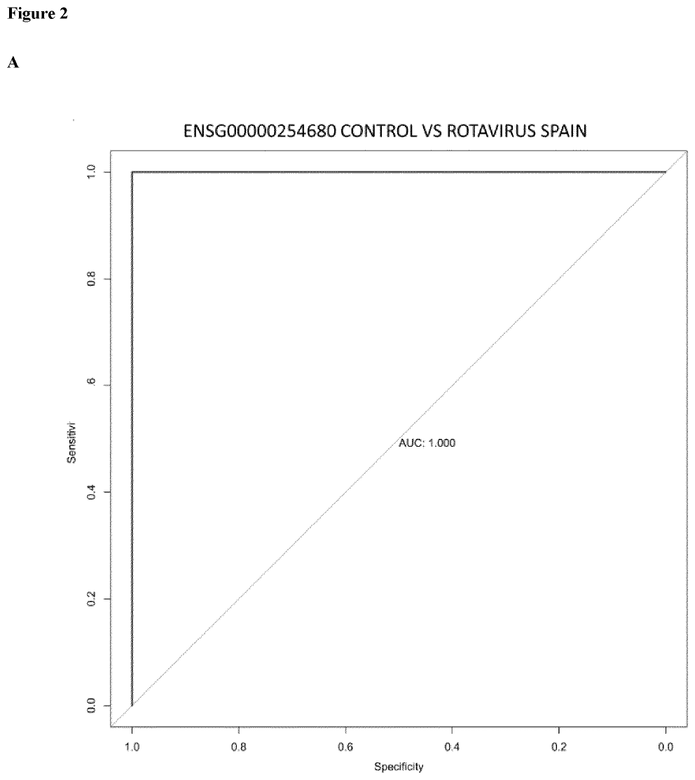

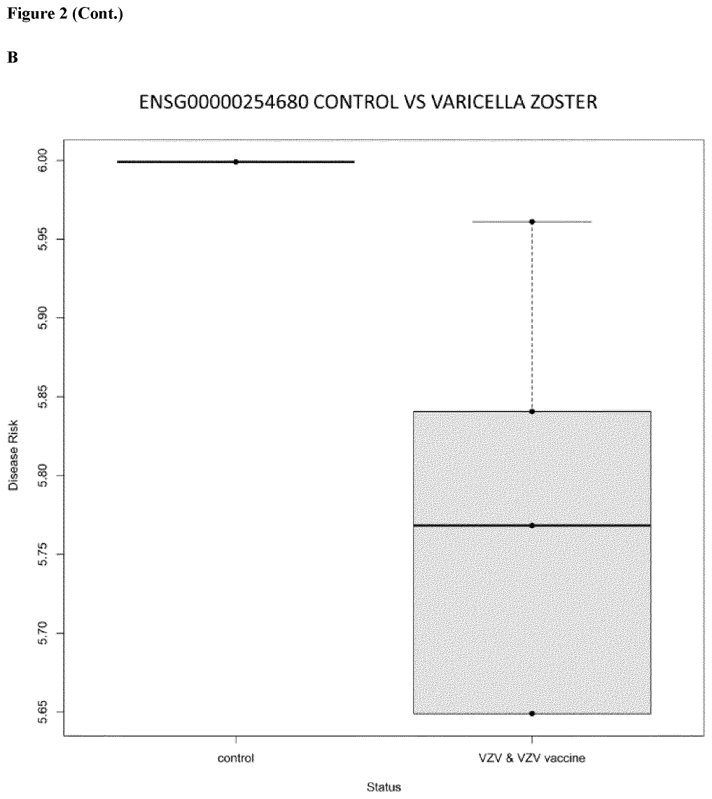

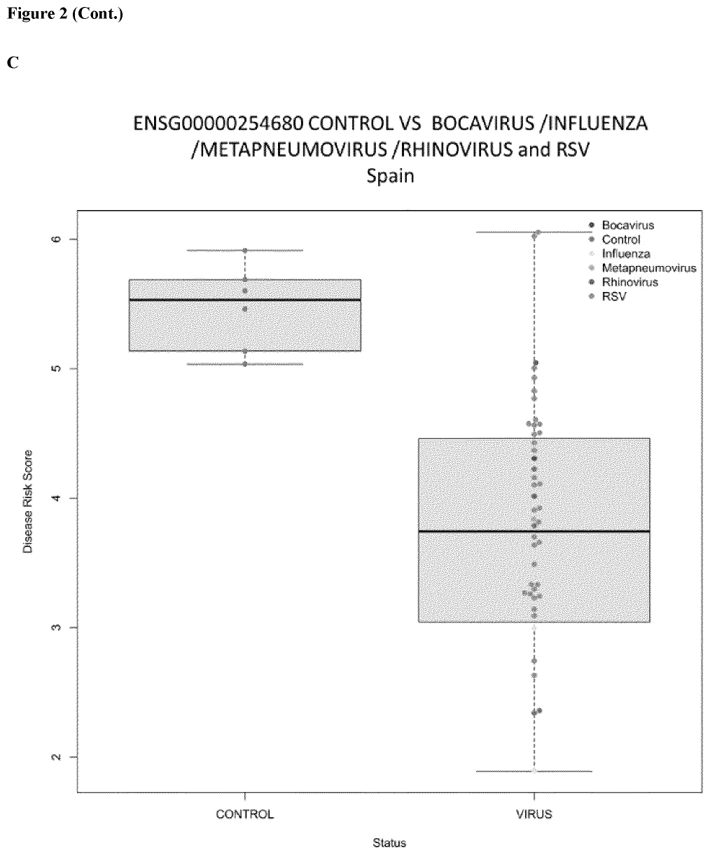

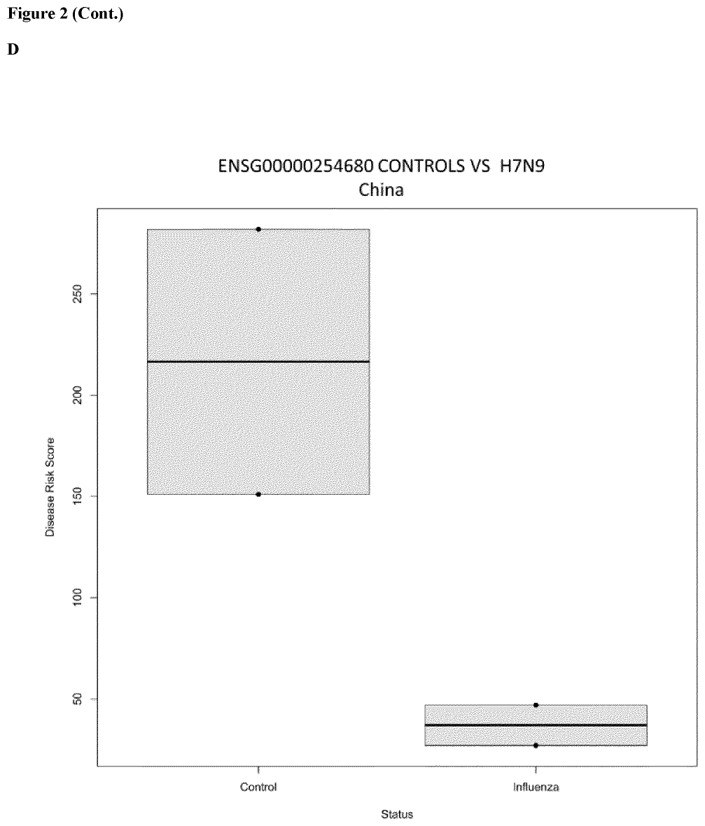

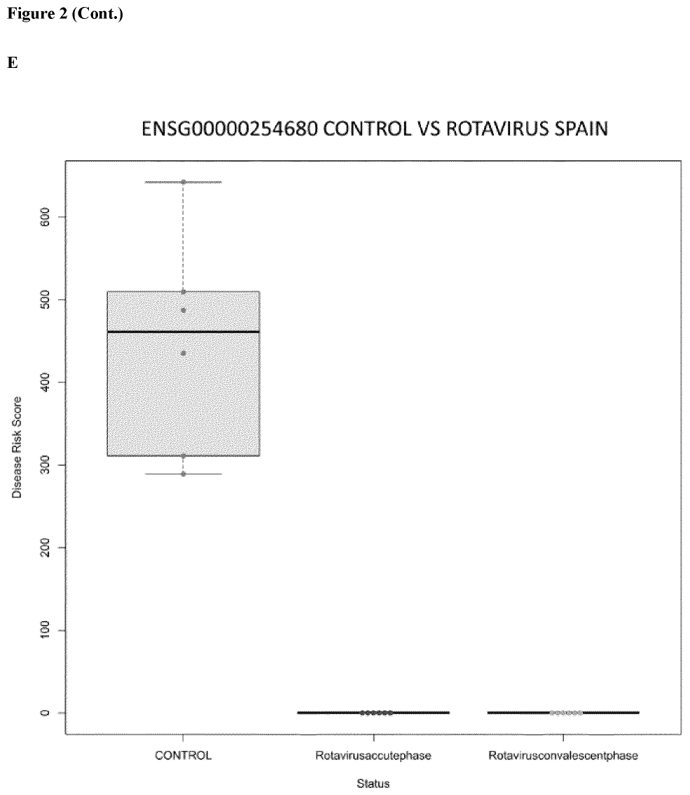

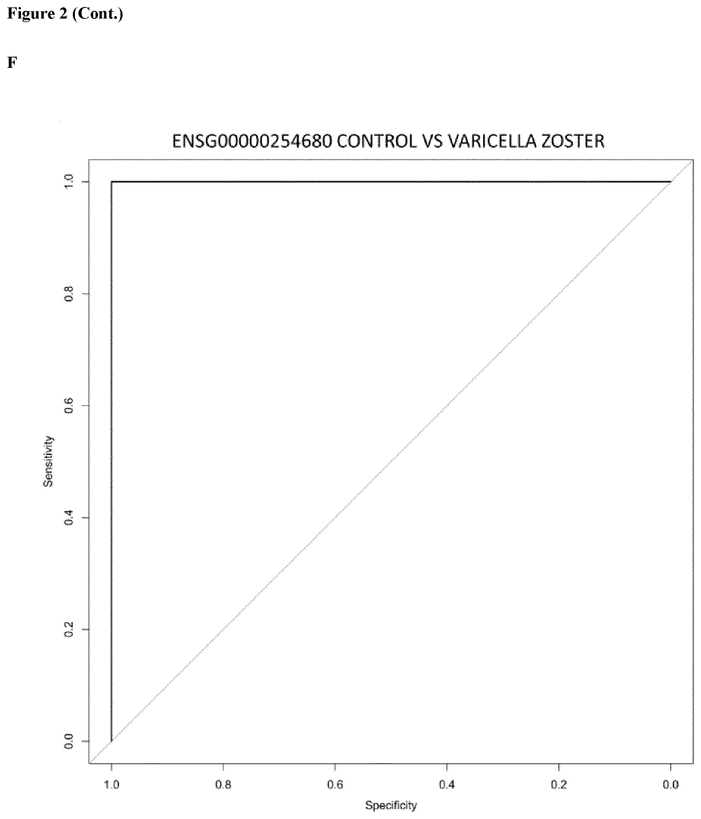

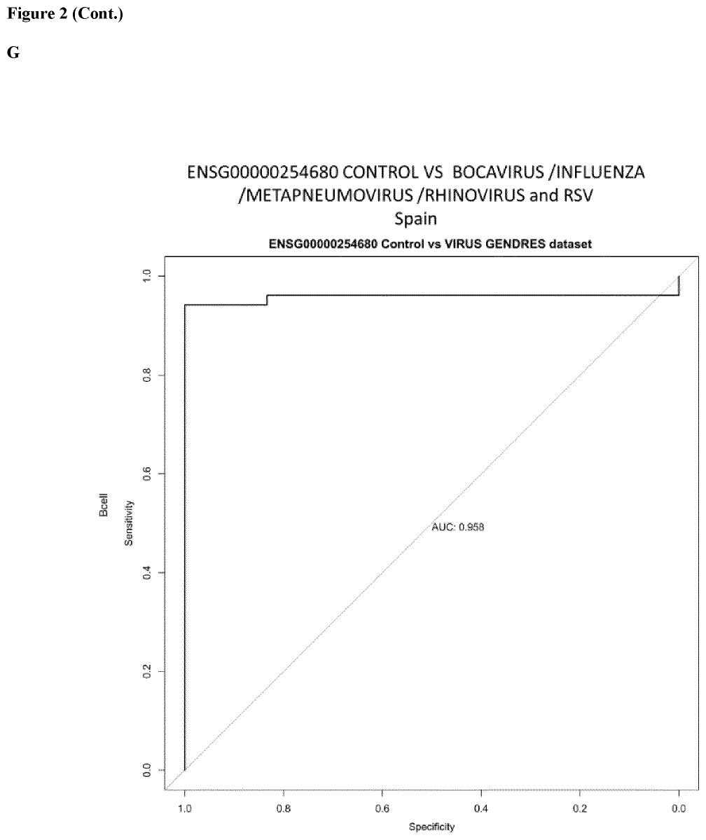

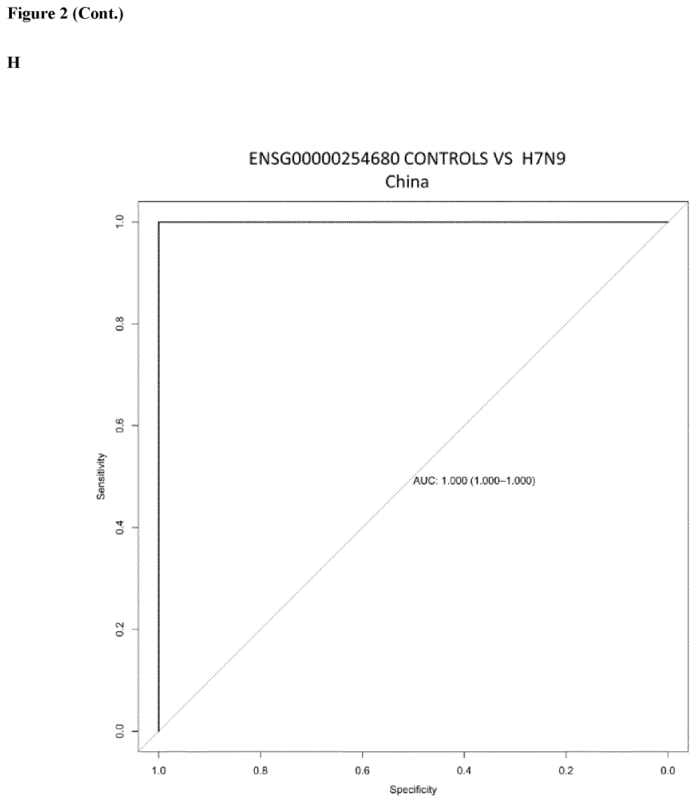

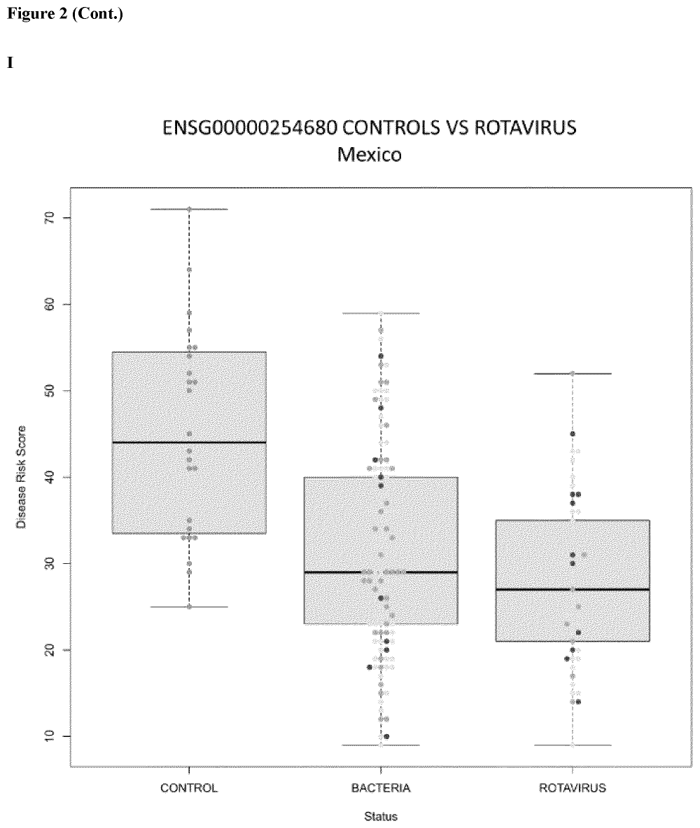

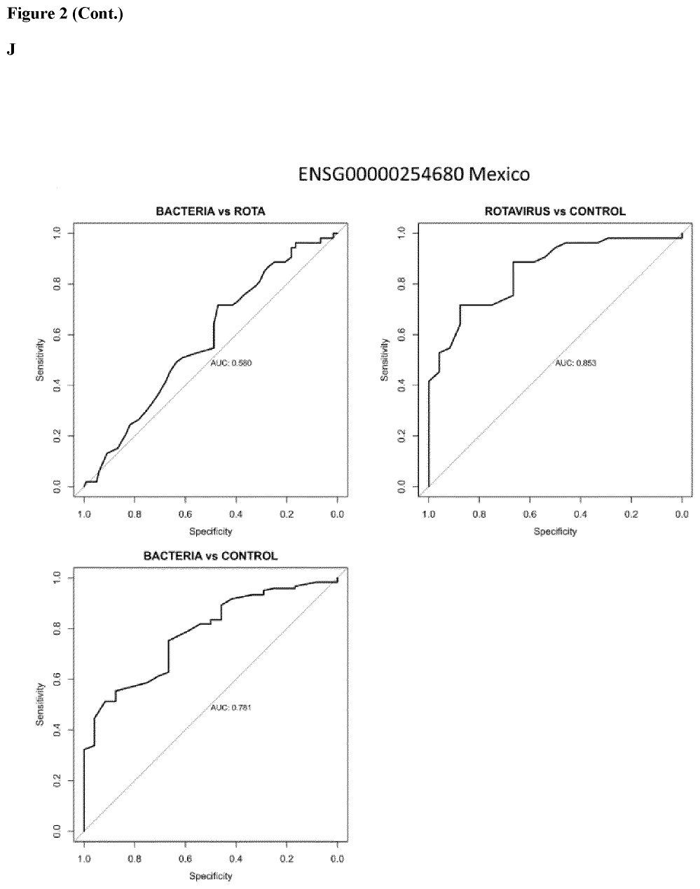

Boxplots were generated with one-dimensional scatter plot with closely-packed but non-overlapping points (FIGS. 1A, 1B, 1C, 1D, 1I, 2B, 2C, 2D, 2E, 2I), which shows a significant difference in the DRS of children affected by viral infection compared to healthy controls. A higher DRS indicates healthy status whereas a lower DRS indicates viral infection.

The diagnostic accuracy of the test to discriminate viral infection was evaluated using ROC analysis (FIGS. 1E, 1F, 1G, 1H, 1J, 2A, 2F, 2G, 2H, 2J), and considering different scenarios: SEQ ID NO: 1 (ENSG00000273149) performance on Rotavirus against healthy control in our discovery cohort (FIG. 1H), SEQ ID NO: 1 (ENSG00000273149) performance on influenza infected versus healthy controls from the study PRJNA230906 (FIG. 1F), SEQ ID NO: 1 (ENSG00000273149) performance on children affected by different intestinal and Respiratory viruses versus healthy controls (FIG. 1J), SEQ ID NO: 1 (ENSG00000273149) performance on Rotavirus infected versus healthy controls and bacterial infected from the study PRJNA285798 (FIG. 1F), SEQ ID NO: 1 (ENSG00000273149) performance on VZV infected epithelial cells versus healthy controls from the study PRJNA497243 (FIG. 1G).

On the other hand, FIG. 2A shows SEQ ID NO: 2 (ENSG00000254680) performance on Rotavirus against healthy control in our discovery cohort, FIG. 2H shows SEQ ID NO: 2 (ENSG00000254680) performance on influenza infected versus healthy controls from the study PRJNA230906, FIG. 2G shows SEQ ID NO: 2 (ENSG00000254680) performance on children affected by different intestinal and Respiratory viruses versus healthy controls, FIG. 2J shows SEQ ID NO: 2 (ENSG00000254680) performance on Rotavirus infected versus healthy controls and bacterial infected from the study PRJNA285798, FIG. 2F shows SEQ ID NO: 2 (ENSG00000254680) performance on VZV infected epithelial cells versus healthy controls from the study PRJNA497243.

For SEQ ID NO: 1 (ENSG00000273149) in all the scenarios, the ROC curve indicates that the accuracy of the test is very high AUC>90% when comparing viral infection from healthy controls. When comparing bacterial vs viral infection, the AUC almost reach the 80% and when comparing bacteria versus controls it drop a little bit to the 76%.

Taken all together, these results suggest that translate this viral signature to a clinical applicable test based on the determination of the level of SEQ ID NO: 1 or SEQ ID NO: 2 may be feasible. Particularly, these results probe that SEQ ID NO: 1 (ENSG00000273149) is the variable with the highest impact in the accuracy of the model based on 2-transcript.

### Example 2.3. 2-Transcript RNA Signature in Virus Versus Controls

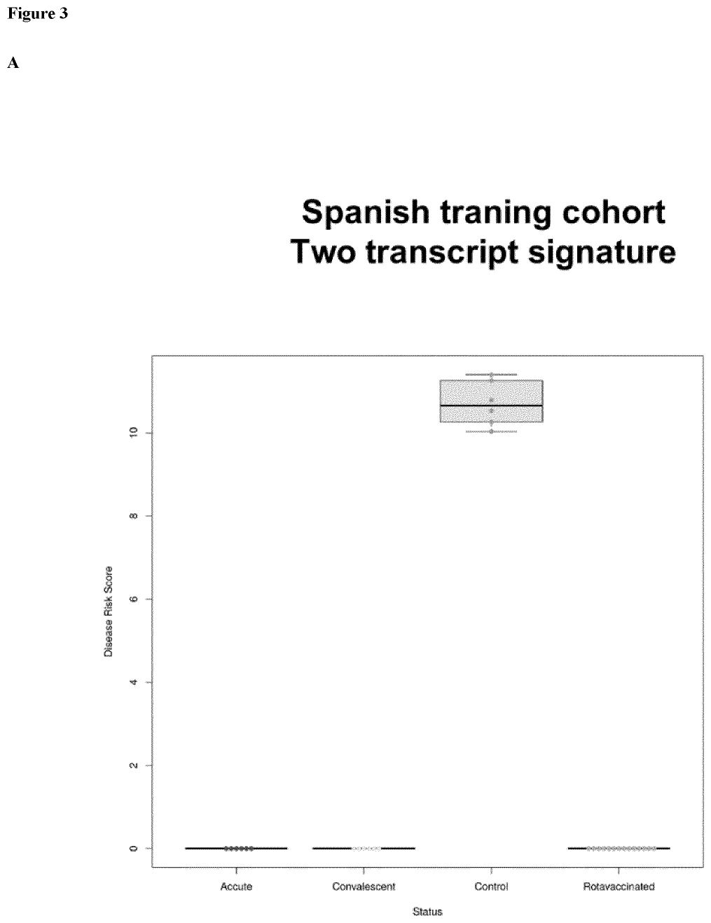

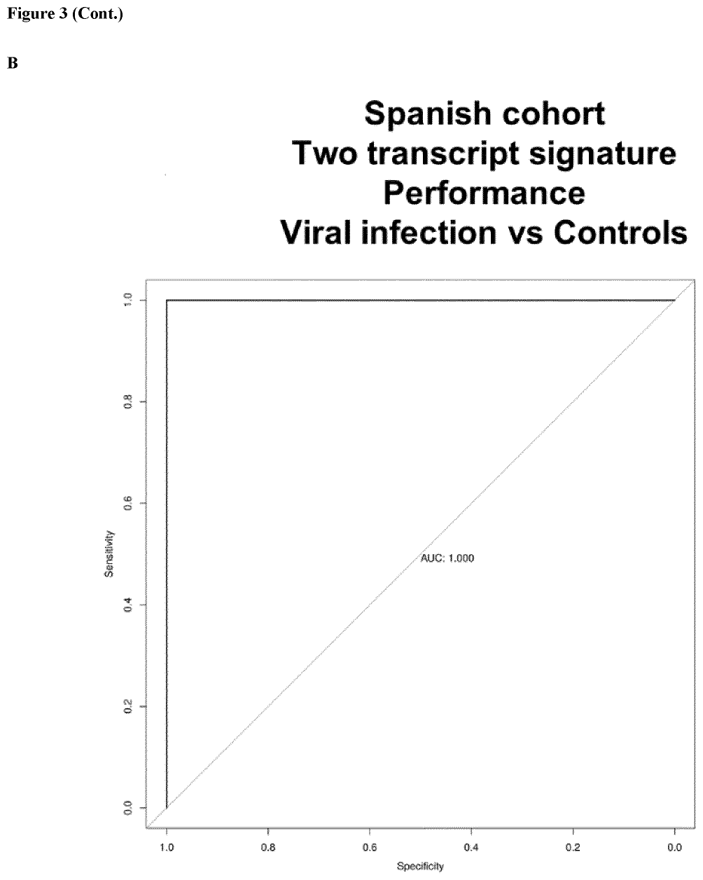

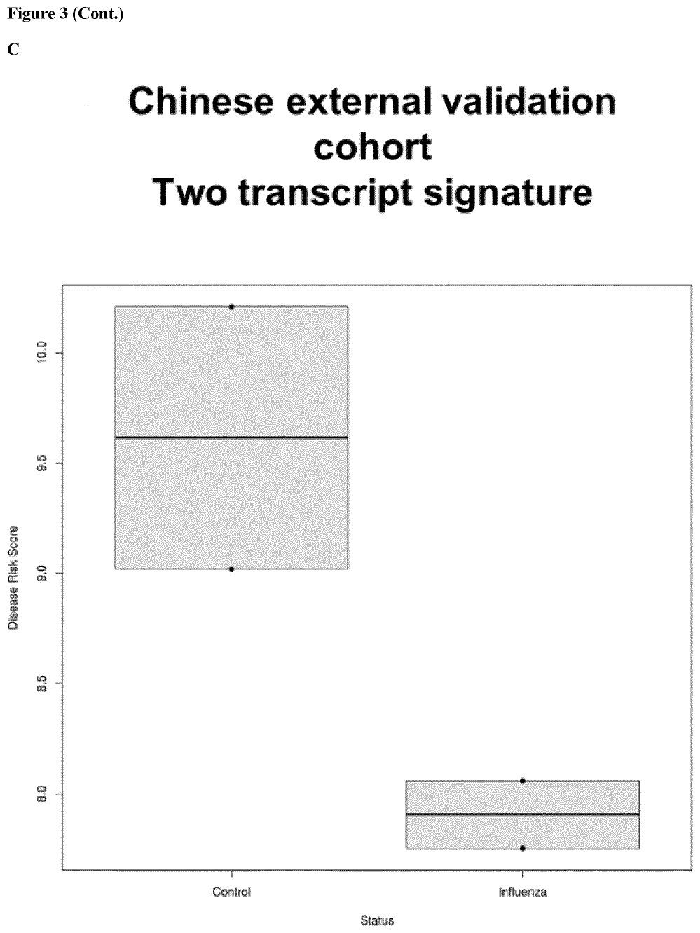

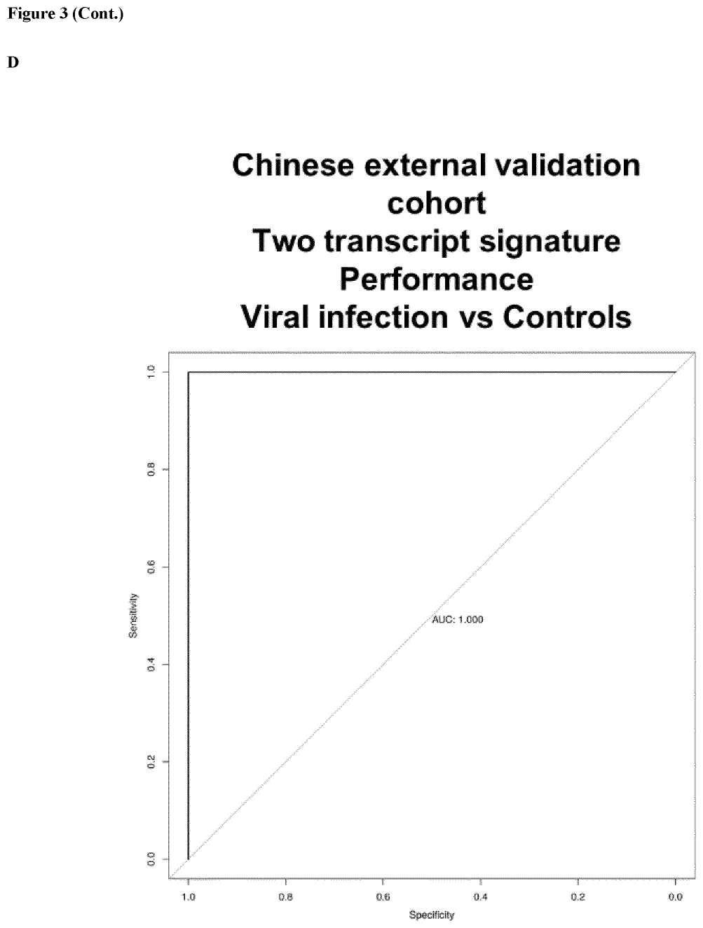

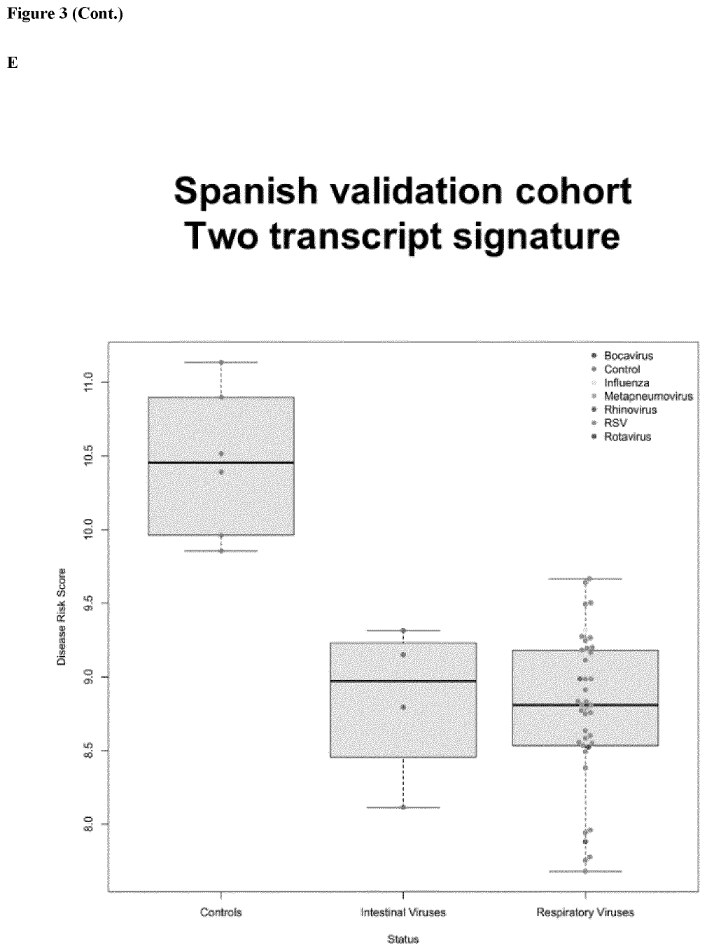

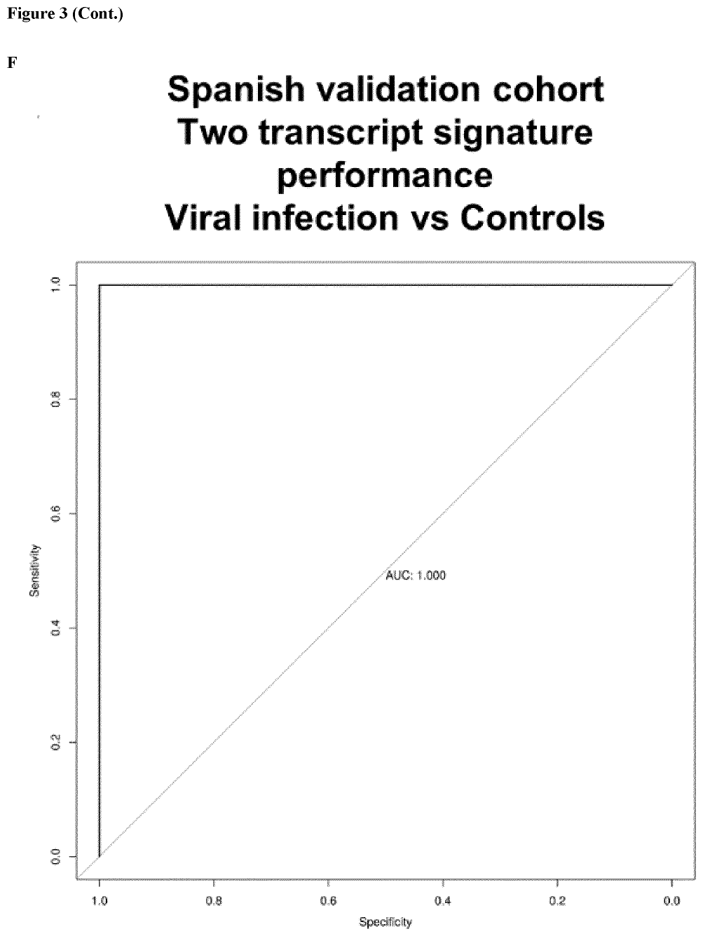

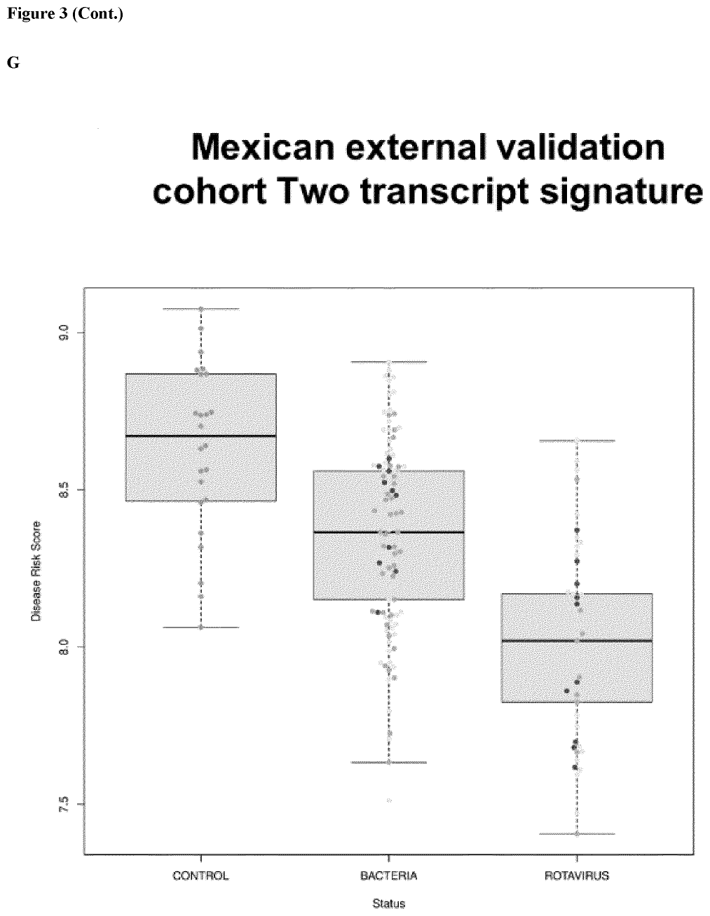

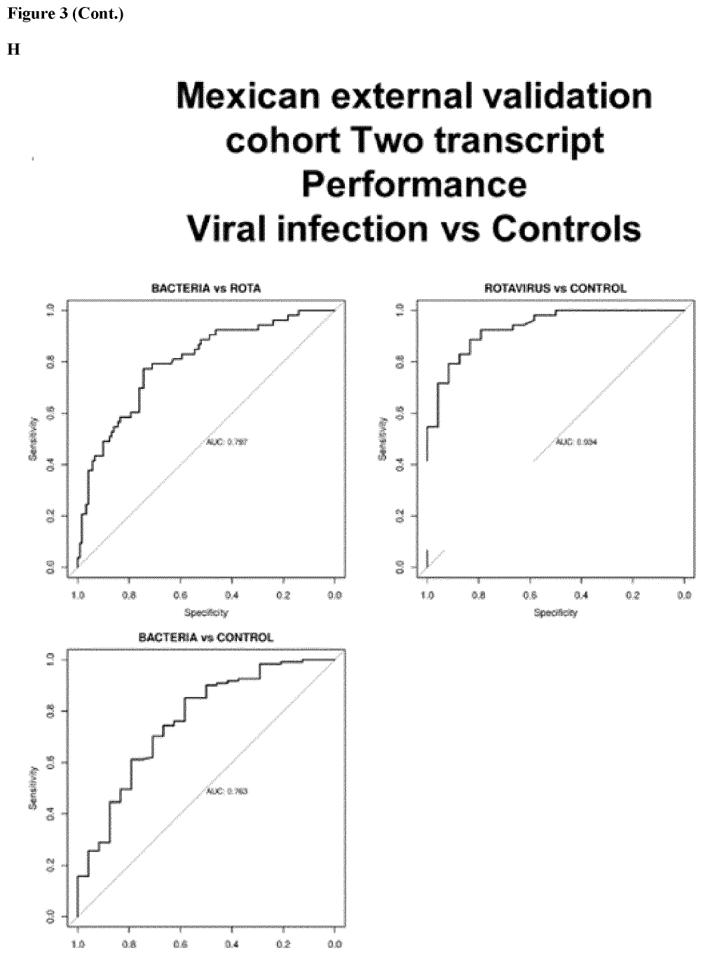

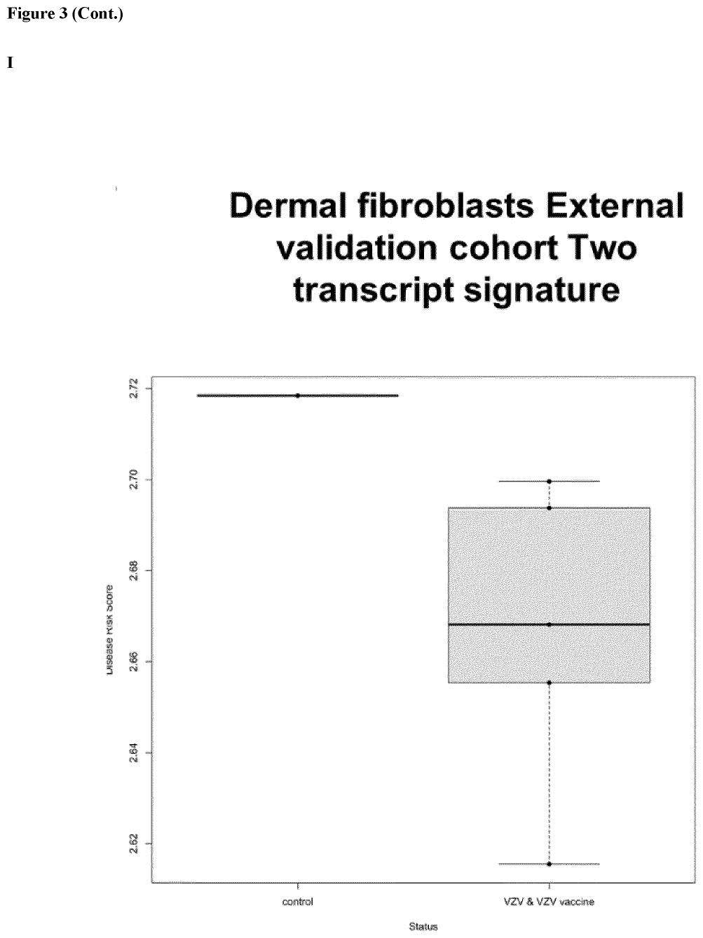

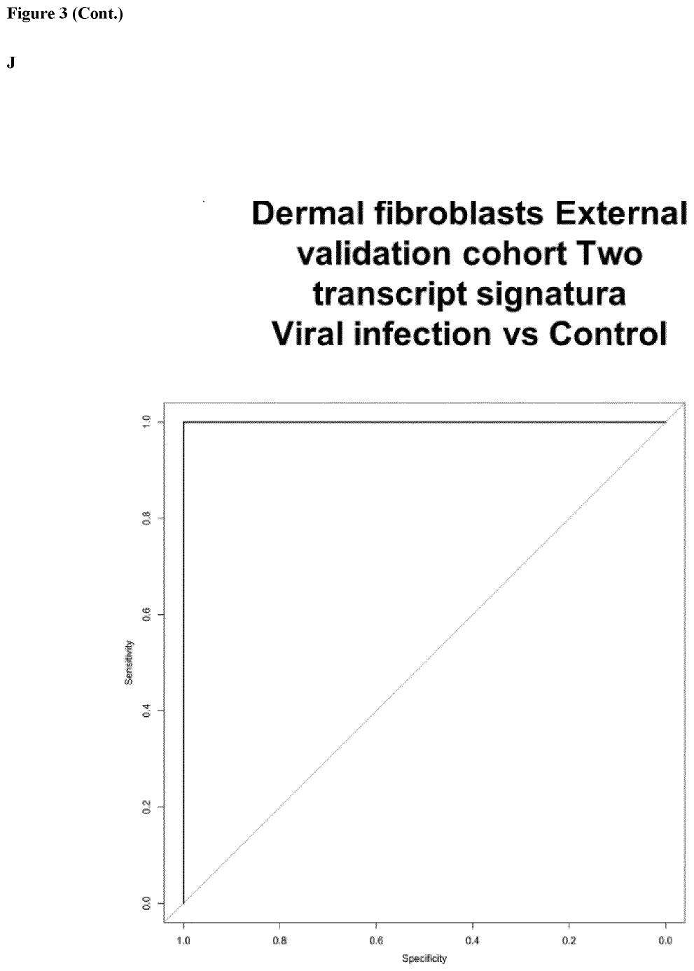

Looking for biomarkers to distinguish vaccinated children from unvaccinated using a Lasso variable selection method followed by a neural network approach, an unexpected but promising result was found. The prediction model based on just two RNAs: Disease Risk Score=log (expression SEQ ID NO: 1)+log (expression SEQ ID NO: 2) can be efficiently used to perform viral diagnose in a broad sense. This model was capable of accurately distinguish between viral infections and healthy controls/bacterial disease in the samples provided in the present invention and four external validation datasets: one from Spain including respiratory and intestinal viruses, one from China with influenza samples (PRJNA230906), one from Mexico (PRJNA285798) with Rotavirus and bacterial samples, and one composed by epithelial cells affected by varicella zoster virus (PRJNA497243) (see FIG. 3).

It was examined whether patients clustered according to their disease status (viral infection, bacterial infection and healthy controls) when applying the DRS. Boxplots were generated with one-dimensional scatter plot with closely-packed but non-overlapping points (FIGS. 3A, 3C, 3E, 3G), which shows a significant difference in the DRS of children affected by viral infection compared to healthy controls. A higher DRS indicates healthy status whereas a lower DRS indicates viral infection.

The diagnostic accuracy of the test to discriminate viral infection was evaluated using ROC analysis (FIGS. 3B, 3D, 3F, 3H, 3I), and considering different scenarios: (B) Rotavirus against healthy control in our discovery cohort, (D) influenza infected versus healthy controls from the study PRJNA230906, (F) children affected by different intestinal and Respiratory viruses versus healthy controls, (H) Rotavirus infected versus healthy controls and bacterial infected from the study PRJNA285798, (I) VZV infected epithelial cells versus healthy controls from the study PRJNA497243. For all the scenarios, the ROC curve indicates that the accuracy of the test is very high AUC>90% when comparing viral infection from healthy controls. When comparing bacterial vs viral infection, the AUC almost reach the 80% and when comparing bacteria versus controls it drop a little bit to the 76%. Taken all together, these results suggested that translate this viral signature to a clinical applicable test may be feasible.

1. In vitro method for the diagnosis of viral infections in a patient which comprises determining the level of at least an RNA of SEQ ID NO: 1, or a protein encoded thereof, in a biological sample obtained from the patient, wherein a reduced level of at least the RNA of SEQ ID NO: 1, or the protein encoded thereof, as compared with a corresponding predetermined threshold level selected to provide a sensitivity and specificity of at least 0.8, is an indication that the patient is suffering from a viral infection.

2. In vitro method for selecting a therapy for a patient which comprises determining the level of at least an RNA of SEQ ID NO: 1, or a protein encoded thereof, in a biological sample obtained from the patient, wherein a reduced level of at least the RNA of SEQ ID NO: 1, or the protein encoded thereof, as compared with a corresponding predetermined threshold level selected to provide a sensitivity and specificity of at least 0.8, is an indication that the patient is suffering from a viral infection and consequently a treatment with antibiotics can be discarded.

3. In vitro method for monitoring the response of vaccinated patients to a viral vaccine which comprises determining the level of at least an RNA of SEQ ID NO: 1, or a protein encoded thereof, in a biological sample obtained from the patient, wherein a reduced level of at least the RNA of SEQ ID NO: 1, or the protein encoded thereof, as compared with a corresponding predetermined threshold level selected to provide a sensitivity and specificity of at least 0.8, is an indication that the patient is responding to the viral vaccine.

4. In vitro method, according to claim 1, wherein the viral infection is caused by Rotavirus, Varicella, Bocavirus, Influenza, Metapneumovirus, Rhinovirus or Respiratory syncytial virus.

5. In vitro method, according to claim 3, wherein the viral vaccine is a vaccine for the prophylactic treatment of a viral infection caused by Rotavirus, Varicella, Bocavirus, Influenza, Metapneumovirus, Rhinovirus or Respiratory syncytial virus.

6. In vitro method, according to claim 1, which comprises determining the level of at least the RNA of SEQ ID NO: 1 in combination with the RNA of SEQ ID NO: 2, or proteins encoded thereof.

7. In vitro method, according to claim 1, wherein the biological sample is blood, serum, plasma or dermal fibroblasts.

8.-13. (canceled)

14. A kit comprising reagents for the determination of the level of at least an RNA of SEQ ID NO: 1 for the diagnosis of a viral infection, and instructions for selecting a therapy for a patient with a viral infection or for monitoring the response of vaccinated patients to a viral vaccine.

15. The kit, according to claim 14, comprising reagents for the determination of the level of at least the RNA of SEQ ID NO: 1 and SEQ ID NO: 2 and directions for the diagnosis of a viral infection, for selecting a therapy for a patient with a viral infection, or for monitoring the response of vaccinated patients to a viral vaccine.


SEQUENCE LISTING (2 sequences)

SEQ ID NO: 1 - DNA, 2345 bp, Artificial sequence
  Synthetic sequence used for identification of viral infection
ttttgctcttgcagttaaaattgtacaatcctttgatccagatacttaaggtatttacctggtagtttttgaaattagcaaggatgtgcttgatttgttctgcagcccctgtcataaaaggttttactctttctggtctctgttcttcaagtttccctttgattctaaaacaacatttcattaacaggttgaagtattgaattttcagtaaaatcaatttttaaagtaaaaaatatccaagcttaaaaaaagaaaacactgaaaaaggcaaccagctgttaagaaattactagttcacagaaggtatctttcaagaatgtttacatttcttgttgactattttcaaaatactattctaattctaaacggaaacagactgcaggcttcagtatgctcatattatagaaaagcaaccactcttttccgtgaactcattaataaaaaagcaaggactttcacaaaagtatacccactgcgaaagaacctcaaaagttagccaatctttaaatcctgtaagattctagacatcagctaggtactgccagtatcacttacgatttcatgtaatctttgatgtacttcttgtaggcttcttttgtgaaacttgtttcctgcaggtgatggttcatgacaatatcgacaccagtgattactgtgctttcggtaccttcgccctcggggccttcagcggaggcatttccaccaatgagcgagtcatcaatgttaccttctgtcctactgaccatctgcattaagaaaaagcagtataattgcaaaagacacaaacgcatccaaagcaccttccacgccaatagttcacggataagaagtattcttagttcttgctgaaaataaaaaaagcaccattttgggaataaagttgtgacccgtggatttctcaaaacgacctaacttaaaagagttgtctgttggccggaacaaaaaatggggtcattaaaaagttgtttccagttgcgggcatgtgatgcctccggttggtaagtggggacaggacaaacacgaaaggactccaggctcctaagccggaagcatcatgtcccggacaaccccgggaggaggatgggcgccgaggcggcgggcctatttccaggatcagctccgcctccagttttctagaaaaacccaagcccggaaagagcgtctcctccgccaggaggcggcggggaggattgcacaacctcaggcgggggagcggaggaaacccgagcccgctcggcccggactcccccacgcgcaggcccgaccgactcaccttcccctccacctccaggcacaacccgtccgcgatctcccggatcttgtagatgtcggagaacatctcatcgtctgccggatacacagagccgcccatcaccgtgcgcctggcgccgccatttcccgcatttcccgcgccggcggccgcacgcgggatctgcccctccgtagcacaccagagctgggcgcgagccccgggcaccgacccctccgcgctcggctaagaccgccggcgtcccctaggcccgcgacggctgcgccttccccgcccccacccgcaccccagacccccgtgtgcggcagtaaggatagtgcagtgaggactcacggctgatgaggtcccggtagataatcatgatggcgactgaagggagacgacgacggcgctagcttagcacgagcctgaaactcggagcgagcgcggtgcagccggagcggcgctcggggggaggggggagcgggcggaaaaggccgactcagccgctccccaacctcatatagagggcacgtccctctacgtcatcgcccgctgcgcctccggaagcgacgcaggcggtgacgtggcacgaagagccgcgcgggggcggggccacagcgggggcgagtcggggaggtgagcatccggggacttgaggcggagacacaaggctccgttccactgggcacgccagcgggtaggggtgggggcggggaggtgcgcgcgcagcgtgggaggaggccaactctcggggcgctccctagccctgggtgggtggagcgggggaagcggagtacgttggacccggctccgccctaccccacggggccgcctggagaggtgctgggagctgggtggagcttagaggaattaaactttggccctgcgcctcgtccagcctaggttccacccttttctgggaacgtgagtatcaaccaaggtaggagaacgtcgcatttagggagggtcaggccctctggcctggggtagatctcgggactgcccgagggcctctgacaacttttcagcatctgccccagggctgccccagctggagtttttgggacgcttttgtgggggcaccgctgagtcc

SEQ ID NO: 2 - DNA, 2948 bp, Artificial sequence
  Synthetic sequence used for identification of viral infection
aatatttatttccatttacagcatcaaataccataaataaagctaaacattgtttgttttgctgtgtgcaaaatacagggttccgtgtggcactgtaaatgattaccaaaggtcacagacatgcgttgtgatgagatggggaggctagctaaagaggaggctgagaggggaggagggcactctttggttgacattctataaagggagggagactctgtaagagaagataagacaagagcaacgactttgtactggccaggatctgcacaaatacagtgagcgtgcagcatgcagggccaggcaggcaggcccgggggtcagggtggtctgggccacatgcaggacagacgcccaccgcagcctgagaccgcgagtgttgcctaggacagctccacggtgcttggtgtgttgtttctcgttaagagtgtgttggtgattatcctgctgccccaggtacaagagtcagcagaaatgtgtgctttaagcagagtcacaggggcctggggctgaactgagtcatttctcaaagatatccctgcctgggatgatgatggctctaattgaagctctggcatcatctggggctttatgagccaagggagataagaagagccacagcaaaacccttgggtctacagtgcaggctgcaaccaaggcagcatttgctagaatatttgtgattatgtgttcaacctacagagcatgtgtcaagcttgggatgaggcctgagagtaattaagttttttaaatccctccaaataatcacctatggttttgataaatgacattaacaatctttacattagagccaatttaagaaggcgctggtatgtatgagctttttgaggctgctcaaaaagtgttcttttcaaaggtgacagaggccccctcatggtattgtttcatgatcaaggtcgaccctcccaagaaagtaggtagcttcgcagacgcaactggagaagcatgcacgcaggcagacccaagggagagaggagaggcggggcatgcacttggggtagaccaggctgccagctccatgggaaatgaacacaggcatgggtctcaagctgaacaccccccgccatgctgttagccaaccatgccaccaaatgagtccatcagtatgcaagacactcacagttagtgcaaccagtactgctggcggagaggacggggtgcggaaagcgggttgggggttagtaccactctttgaaaattggcaagaaaaccagtcaccttagagcagaagtgtgtcagccaagaagtgggtgtagcactggaaggctgaaatgaactgatggccatggcgtaagattggaaagagagagaaagatagaaaagagagcttagtgtggggaaaaaaggaaatgattaacatcacgagaaggcaccggagttgaaaataaggccacgattgaaagagggaaacttcctctgaaagagtcgctgtttgcttaggagagatgaacctttctggttgtgaccagctacttgtttacgagagtggatgcacacagtttgcatcttgctccctgatatcctctctgcccccattttactctagcgtctcccattcattcttgaatatgttgcggaggcctagaggcattcatcctcgggggtccacccctgggtgtcggtgctgcccaccagaaaggttcaacatctcagagcgtacaaacatctcagatttctgacaggcacgccctgtctcgccacagtaattcaaaccagtctccttatggctgtacacgacagtcagagattccgactcgggcttcatggaacactccacaaagcatctcagcctatcctgagctgaagataatcacaataacatggccgttcacatcatgacctcaaaaatttactttctatactcaaacaaaaggaatcaagggttcctcccaccatgctgaacattaaaatcccagcaaaaattggagaaaacacaatgcaagtcatcggactgagaacagtgccaacaaggacagaggcgccagttcccacagagttggccacggccatgcctctgtttttcccaaggatttcagggaagtgtttgtagaaatgcacctccttcttcccgccccatagaaactgtcatctcaaagctctcaaaggttctgactcctcacccacacccaggcacacctcttagttaatccagaggcatctctagtgataactccacagagctatcttgggcccagcaactccagatacactgcgcaccgtagatggcctttctctggcggccccttgattgttcagccagaccctgcagctgtgtatgtctaggcagcgtggagtagggagagctgaggactcttctagatacccagagtaagaacacaggtggacctgcatgctcatttttacccttactcatgcatatgtcctttggatcagtccataaatagcttccactcccagccttccctgccagtcaagatatggtagagcttcccctggaggtcagacccattcctttcccagggcagtgtgaaattggtttctgttttgaccacatatgtgtggcagcggagcagctaagcagtcaggatgtccacaggcaatgctcactgggcaggccacgcttcccagcaatagcctgatgtgctaacaaaccccctcagtcccaaaggacaccacataagtggacagagtcttccactccacttggcctgaccaatatggaccctgcacagccttggctcagcctgcagcccgggctgggggatccgctgggagccacaggggctaaaatgccatcagttgaaagagctcatctttggcactgcatcagctccatggtcaaatgccaatgggctttccctgcagtgcccattagtgggaagggaaaatgcatgtttgtagaatctgttgaaaccccacggttctttgtcatttggctgtgagttggtatgggggggaaagcctttaag